(19) United States
(12) Patent Application Publication
(10) Pub. No.: US 2002/0168372 A1
(43) Pub. Date: Nov. 14, 2002

(54) DNA SEQUENCE ENCODING A PAPILLOMAVIRUS L1 PROTEIN CAPABLE OF EFFICIENTLY FORMING VIRUS-LIKE PARTICLES

(76) Inventors: MATTHIAS DURST, JENA (DE); LATZ GISSMANN, WIESLOCH (DE)

Correspondence Address: PENNIE & EDMONDS LLP, 1155 AVENUE OF THE AMERICAS, NEW YORK, NY, 100362711

(21) Appl. No.: 09/162,904

(22) Filed: Sep. 29, 1998

Related U.S. Application Data
(63) Continuation of application No. 08/884,168, filed on Jun. 27, 1997, now abandoned.
Application No. 08/884,168 is a continuation of application No. 08/641,570, filed on May 1, 1996, now abandoned.
Application No. 08/641,570 is a continuation of application No. 08/092,528, filed on Jul. 16, 1993, now abandoned.

(51) Int. Cl. A61K 39/00; A61K 39/38

(52) U.S. Cl. 424/184.1

(57) ABSTRACT

The present invention relates to a DNA sequence encoding a papillomavirus L1 protein capable of efficiently forming virus-like particles (VLP). In particular, the present invention relates to a DNA sequence encoding an HPV16 L1 protein. Furthermore, the present invention relates to expression plasmids containing said DNA, to host cells transformed by said expression plasmids, to methods for the production of said L1 protein, to the VLP formed by said L1 protein, to antibodies reacting with said protein and said VLP, to diagnostic and pharmaceutical compositions and methods and to a vaccine comprising said VLP.

The present invention relates to a DNA sequence encoding a papillomavirus L1 protein capable of efficiently forming virus-like particles (VLP). In particular, the present invention relates to a DNA sequence encoding an HPV16 L1 protein. Furthermore, the present invention relates to expression plasmids containing said DNA, to host cells transformed by said expression plasmids, to methods for the production of said L1 protein, to the VLP formed by said L1 protein, to antibodies reacting with said protein and said VLP, to diagnostic and pharmaceutical compositions and methods and to a vaccine comprising said VLP.

Papillomaviruses are widespread in the animal world and there are representatives infecting different species including fish, birds and a whole variety of mammals such as cattle, rabbits, dogs and man. Virus replication takes place in usually benign epithelial proliferations which develop after virus infection. In some animals there is a well documented link between papillomavirus infection and malignant cancer (e.g. skin cancer in rabbits, see (1); cancer of the bladder or the upper alimentary tract in cattle, see (2); (3)). The human papillomaviruses (HPV) represent a very heterogeneous group of agents. At present almost 70 different types have been published, 30 of which infect the anogenital tract (see 4)). During the last years it became evident that some of these mucosotropic human papillomaviruses such as HPV16, 18, 31, 39 and 52 are associated with malignant tumors of the anogenital tract, in particular with cancer of the uterine cervix. In particular in case of HPV16 there is both epidemiological and experimental evidence in support of a causative role of these viruses in the development of cancer in the uterine cervix. The strongest argument is the regular presence of viral genomes in tumor biopsies and in cell lines derived therefrom as well as the demonstration of transforming activity of the early viral genes E6 and E7 in vitro. Inhibition of E6/E7 oncogene expression by antisense transcripts leads to growth inhibition of cervical cancer cells and to their non-tumorigenicity in nude mice. It is furthermore known that cervical cancer is a multifactoral disease and that additional agents such as tobacco smoke or certain microorganisms may act synergistically towards the malignant transformation of epithelial cells.

Despite the involvement of additional factors, it is generally accepted that HPV infection is most important in the development of cervical cancer. Thus, diagnosis of and interference with HPV infection is of outmost relevance for cancer detection and control. Because of the high prevalence of asymptomatic genital HPV infection, detection of HPV DNA in clinical materials is of limited diagnostic value. However, serology of HPV infections is only poorly developed because of lack of suitable experimental systems for virus replication and production of viral proteins that can be used as antigens in serological assays. This biological feature of papillomaviruses is particularly important in case of the mucosotropic HPV types since their replication in clinical lesions is very low. Thus, preparation of viral proteins and HPV virions in sufficient quantities is not possible at all. Despite recent advances in the propagation of HPVs in mouse xenografts and in raft cultures these systems are not suitable to yield preparative amounts of HPV L1 protein or virions. The conformational dependency of neutralizing epitope, as observed in experimental animal papillomavirus systems (see (5), (6)) suggests that properly assembled HPV particles might be critical for the induction and detection of clinically relevant immune reactivity. For HPV16 it was shown that although expression of the prototype L1 gene (6) alone or coexpression of the prototype L1 gene along with the minor capsid protein L2 did result in VLP formation, the VLP were either significantly smaller than native virions (7) or the efficiency of self-assembly was extremely low (6).

Consequently, the technical problem underlying the present invention is essentially to provide a DNA sequence encoding an HPV16 L1 protein capable of efficiently forming virus-like particles in preparative amounts allowing the detection of HPV16 infection and vaccination against said infection. The solution to the above technical problem is achieved by providing the embodiments characterized in the claims.

Other features and advantages of the invention will be apparent from the description of the drawings and the preferred embodiments which represent the best mode of carrying out the invention. The sequence listing and drawings will now be briefly discussed.

SEQ ID No. 1 shows the nucleotide sequence of the L1 gene of HPV16 DNA clone P114/16/2 (termed in the following as 114/B) deposited on Jul. 14, 1993 at the DSM, Braunschweig, Germany in an E. coli K12 derivative under accession No. DSM 8418.

SEQ ID No. 2 shows the nucleotide sequence of the L1 gene of HPV16 DNA clone P114/16/11 (termed in the following as 114/K) deposited on Jul. 14, 1993 at the DSM, Braunschweig, Germany in an E. coli K12 derivative under accession No. DSM 8419.

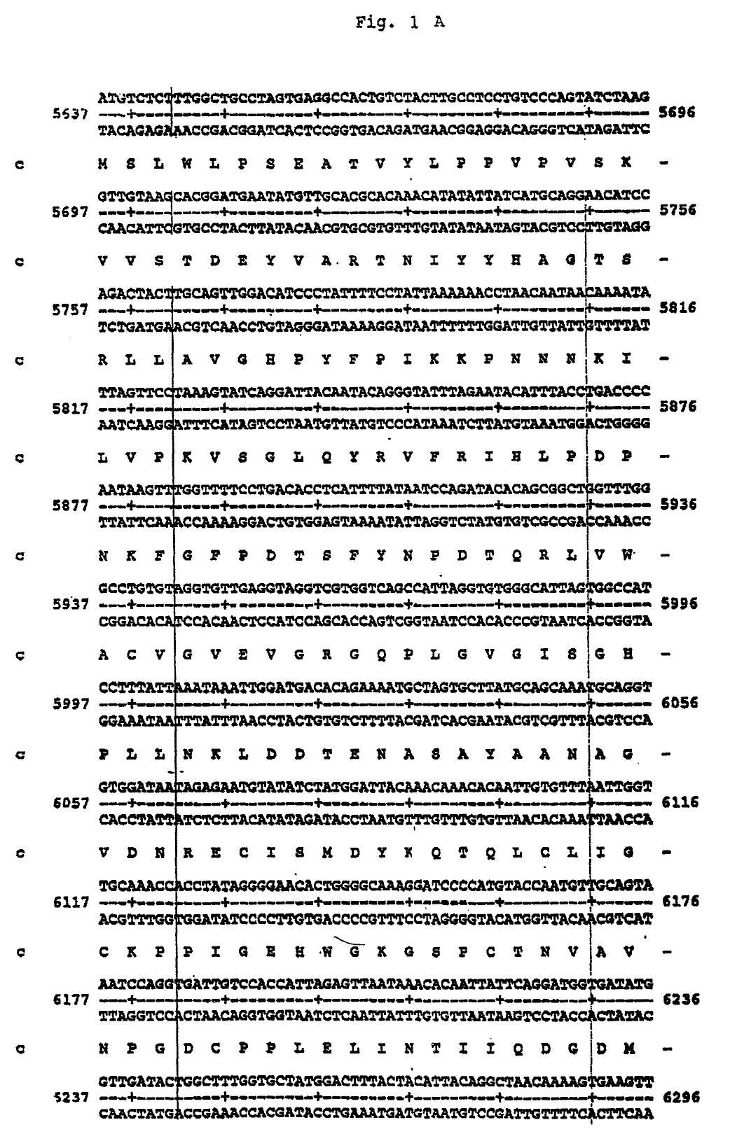

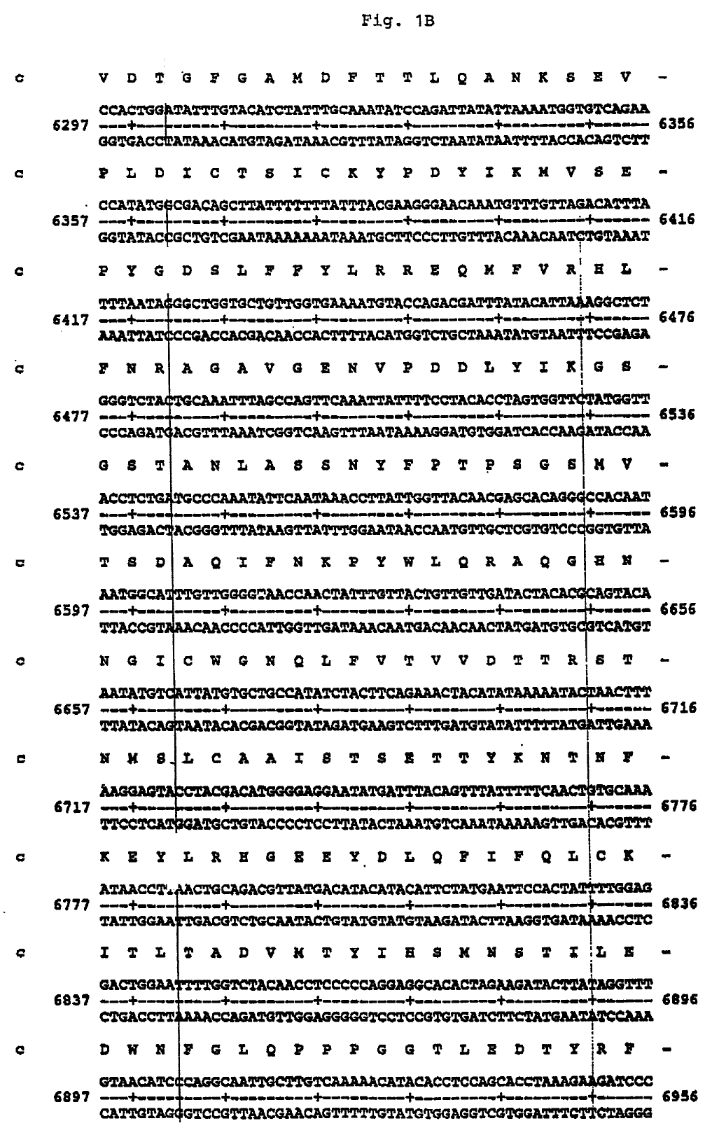

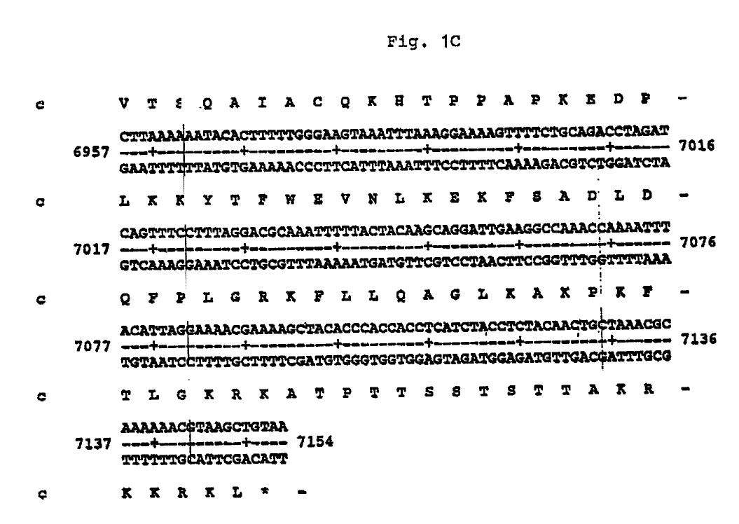

FIG. 1: This figure shows the nucleotide and aminoacid sequence of Clone 114/B.

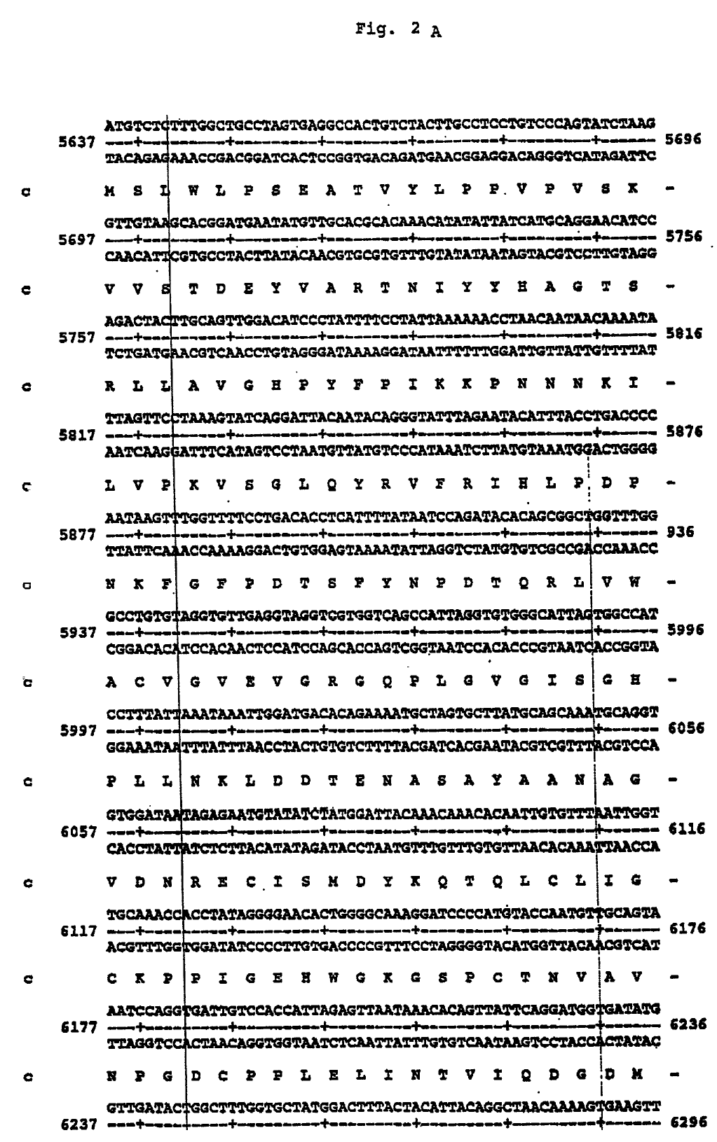

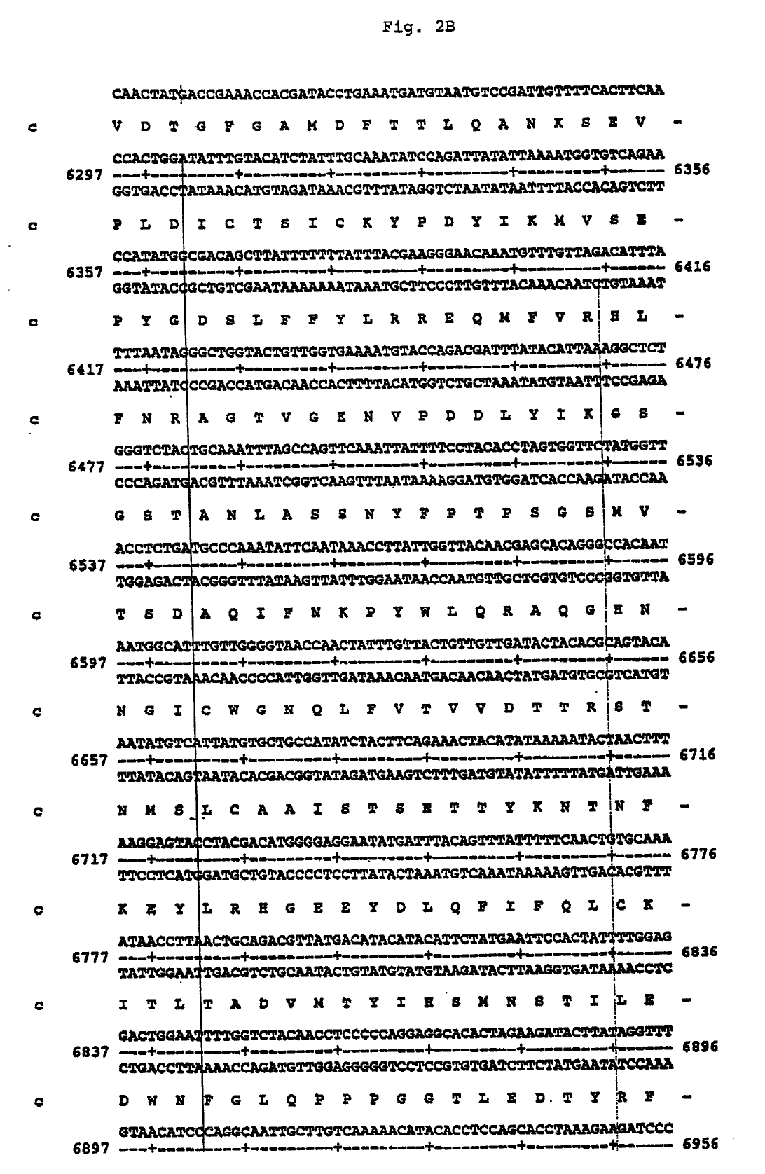

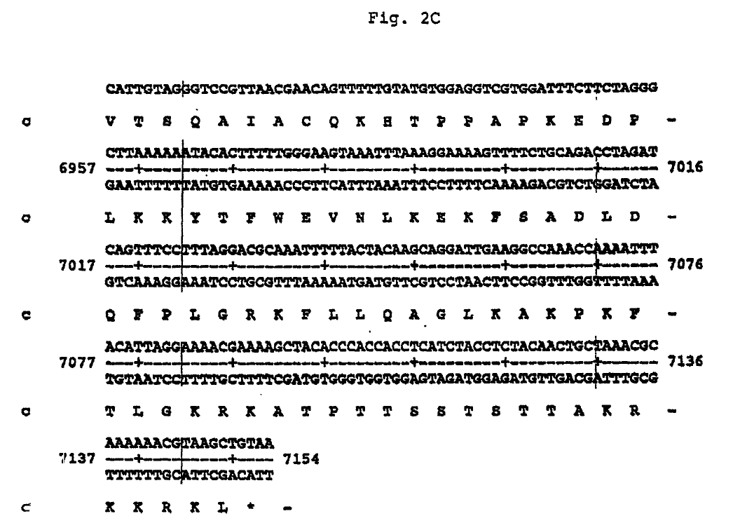

FIG. 2: This figure shows the nucleotide and aminoacid sequence of Clone 114/K.

The present invention relates to a human papillomavirus L1 protein capable of efficiently forming VLPs and provides DNA sequences encoding said protein. Such sequences include in particular the sequences as illustrated in SEQ ID No. 1, SEQ ID No. 2, and DNA sequences degenerated as a result of the genetic code for said sequence. They also include DNA sequences hybridizing with the DNA sequence mentioned above, comprising at triplet 202 a DNA sequence encoding Asp or Glu and being capable of forming VLPs.

Although such degenerate and hybridizing sequences may have structural diversity due to naturally occurring mutations such as deletions, additions, inversions or substitutions, the encoded L1 protein will usually still exhibit essentially the same useful properties, in particular its capability of efficiently forming VLPs in preparative amounts, allowing their use in basically the same diagnostic and therapeutic applications.

The DNA sequence of the invention can be obtained by the present invention. However, in case the obtained DNA sequence differs in some positions from the claimed sequence, said particular DNA sequence can easily be generated by using site-directed mutagenesis on the obtained DNA sequence.

According to the present invention, the term “hybridization” means conventional hybridization conditions. The term “hybridization” preferably refers to stringent hybridization at Tₘ −20° C. and washing conditions at Tₘ −20° C., most preferably to stringent hybridization at Tₘ −10° C. and washing at Tₘ −10° C.

Preferred embodiments of the present invention are DNA sequences as defined above and obtainable from human papillomaviruses, in particular HPV16.

Particularly preferred embodiments of the present invention are the DNA sequences as shown in SEQ ID No. 1 and No. 2. In comparison to the prototype L1 DNA sequence, the only difference in the entire L1 open reading frame of clone 114/K (SEQ ID No. 2) is a single nucleotide 6240 (corresponds to nt. 604 in SEQ ID No. 2) C to G base change which results in a non-conservative change of Histidin to Aspartat at amino acid 202 (Table 1). Therefore, this single amino acid difference is responsible for the inefficient self-assembly of the prototype L1 protein. Clone 114/B (SEQ ID No. 1) also encodes Aspartat at amino acid 202. This latter clone contains two additional amino acid changes with respect to the prototype L1 protein: Val 194 to Ile and Thr 266 to Ala (Table 1). Amino acid sequence alignment of L1 open reading frames of different human and animal papillomaviruses indicates that aspartic acid or glutamic acid is present in all of them at amino acid position 202 and the various L1 proteins found to efficiently self-assemble each encode one of these two amino acids at this position.

As is evident from the following, the present invention relates to the preparation of host cells capable of producing the L1 protein, the production of said protein, the self-assembly into VLPs and the production of antibody, in particular monoclonal antibodies specifically reacting with said L1 protein and said VLP. Said objects are easily accomplished using known recombinant DNA techniques comprising constructing the expression plasmids encoding said protein and transforming a host cell with said expression plasmids, cultivating the transformant in a suitable culture medium and recovering the L1 protein. Thus, the invention also relates to recombinant molecules comprising DNA sequences as described above, optionally linked to an expression control sequence. Such expression control sequence may also include inducible expression control sequences. Several animal, insect, plant, fungal and bacterial systems may be employed for the transformation and subsequent cultivation process. Preferably, expression vectors which can be used in the invention contain sequences necessary for the replication in the host cell and are autonomously replicable. It is also preferable to use vectors containg resistance genes which allow selection for transformed host cells. The necessary operations are well known to those skilled in the art.

It is another object of the invention to provide a host cell transformed by an expression plasmid of the invention and capable of producing an L1 protein. Examples of suitable host cells include various eucaryotic and procaryotic cells such as bacillus or E. coli, plant cells such as tobacco, potato or arabidopsis cells, animal cells such as incect cells or mammalian cells, preferably cells of the Mo−, COS−, C127− or CHO− cell line and fungi such as yeast.

It is a further object of the invention to provide a process for the production of an L1 protein capable of forming VLPs with high efficiency. Such a process comprises cultivating said host cells being transformed by a DNA sequence of the present invention in a suitable culture medium and purifying the L1 protein produced. Thus, this process will allow the production of a sufficient amount of the desired protein for use in medical treatments or dignosis. Due to the nature of recombinant DNA technology, it will be understood that the protein as obtained by said process is free from polypeptides, proteins or hormones with which it is naturally associated. Furthermore, depending on the host cell, the protein of the invention can be free from human, mammalian, bacterial, fungal, viral or plant proteins or substances.

A further object of the present invention is to provide an L1 protein of a papillomavirus capable of forming efficiently into VLPs. In particular, said protein is encoded by a HPV16 genome. Particularly preferred embodiments of said proteins are the proteins encoded by the DNA sequence given in SEQ ID Nos. 1 or 2. DNA sequence comparison between the HPV16 prototype L1 gene and the both preferred embodiments of the present invention revealed a single non-conserved amino acid change into an acidic amino acid (Glu or Asp) at position 202 to be responsible for the efficient self-assembly of the L1 proteins of the present invention into VLPs compared to the inefficient self-assembly of the prototype L1 protein. In particular, the present invention provides HPV16 L1 proteins assembling into VLPs with at least three orders of magnitude higher efficiency than the prototype L1 protein, although in insect cells similar levels of L1 protein are expressed from the prototype L1 gene and the both preferred embodiments of the present invention as shown in SEQ ID No. 1 or 2.

The present invention also relates to the VLPs comprising the L1 proteins of the present invention, optionally in conjunction with an L2 protein. The term VLP as used in the context of the present invention refers to a VLP formed by an L1 protein encoded by the DNA sequences of the present invention and modifications of said VLP as long as said modifications exhibit essentially the same immunological or biological properties.

It is another object of the present invention to provide antibodies which specifically react with an L1 protein encoded by the DNA sequences of the present invention. A preferred embodiment of the present invention relates to monoclonal antibodies directed specifically to an L1 protein encoded by the DNA sequences of the present invention. In the context of the present invention the term “directed specifically to an L1 protein” means that said antibody specifically binds to the L1 protein encoded by the DNA sequences of the present invention but not to the known L1 proteins such as the prototype L1 protein derived from HPV16.

It is a further object of the present invention to provide antibodies which specifically react with a VLP comprising the L1 protein of the present invention, optionally in conjunction with an L2 protein. A preferred embodiment of the present invention relates to monoclonal antibodies directed specifically to a VLP of the present invention comprising the L1 protein of the present invention, optionally in conjunction with an L2 protein. In the context of the present invention the term “directed specifically to a VLP of the present invention” means that said antibody specifically binds to the VLP of the present invention but not to the VLPs known in the prior art.

It is another object of the present invention to provide pharmaceutical and diagnostic compositions or kits containing a therapeutically or diagnostically effective amount of the L1 protein, the VLP or the antibody of the present invention. Optionally, such a composition comprises a pharmaceutically acceptable carrier and/or diluent. The present invention provides the method and means to generate preparative amounts of VLPs, in particular HPV16 VLPs which may prove particularly useful for the development of a sensitive serological assay to measure HPV16 virion antibodies in mammals, particularly in humans. Accordingly, the present invention relates to a diagnostic kit for the measurement of anti-HPV16 virion antibodies in a sample comprising the VLPs of the present invention as an antigen. Furthermore, the present invention relates to a method for determining anti-HPV16 virion antibodies in a sample comprising the use of the VLP of the present invention as an antigen.

Furthermore, the present invention provides the means and methods for the production of preparative amounts of VLPs, in particular HPV16 VLPs which prove particularly useful for preparing an effective pharmaceutical composition comprising said particles optionally in conjunction with a pharmaceutically acceptable carrier and/or diluent. In particular, said pharmaceutical composition is a vaccine. More particularly, said vaccine is a vaccine against papillomavirus infections, preferably HPV16 infection. The vaccine comprising the VLP of the present invention can also be used prophylactically. Furthermore, the application of the composition is not limited to humans but can also include other mammals.

The following Examples illustrate the invention but should not be construed as limiting the invention.

### EXAMPLE 1

### Construction of L1 and L2 Recombinant Baculoviruses

Three L1 gene expression vectors each containing an L1 gene were constructed, one contained the prototype L1 gene for comparative purposes and the both other vectors contained two newly isolated L1 genes. In particular, single gene baculovirus transfer vectors were constructed which contain the L1 coding region (nt. 5637-7154 (corresponds to nt. 1-1518 of SEQ ID No. 1 or 2)) of two HPV16 DNA clones isolated from condylomata acuminata. Using the cloning strategy previously described for L1 from the prototype HPV16 clone (6) two clones were obtained, 114/K and 114/B. These constructs were made by cloning each L1 gene into the expression vector pEVmod, resulting in a L1 gene being under the control of the polyhedrin promoter. The prototype L2 ORF which encodes the minor HPV capsid protein (nt. 4235-5656) was also placed in this vector. In addition, HPV16 L1/L2 double expression vectors were constructed, using pSynwtVI, such that the prototype HPV16 L2 gene was expressed under the control of a synthetic promoter pSyn and the HPV16 L1 genes (prototype, 114/K, 114/B) were expressed under the control of the polyhedrin promoter. Both above-mentioned expression-vectors are publically available from (8). The L1 genes were cloned as 5′BglII to 3′BglII fragments into pEVmod or pSynwtVI. For cloning L2 into pSynwtVI, the L2 primer sequences were:

(sense) 5′-
GCGGTGATATCAATATGCGACACAAACGTTCTGAAAACGCACAAAACGT-
3′;
(antisense) 5′-
CCGCTCCGCGGACTGGGACAGGAGGCAAGTAGACAGTGGCCTCA-3′.

Restriction sites (underlined) were included in the oligonucleotide primers and used for cloning L2 as a 5′ EcoRV to 3′ SstII fragment into the baculovirus double expression vector. For cloning L2 into pEVmod, comparable primers with BglII restriction sites at both ends were used, so that L2 was cloned as a 5′BglII to 3′BglII fragment. Sequencing was carried out by conventional methods for the vector/HPV junctions and, for HPV16, the entire L1 and L2 ORFs of the baculovirus vectors. Numbering refers to the prototype HPV16 sequence, GenBank accession #K02718, which is not corrected for the missing t at nt. 3903 in the E5 ORF (9) and for the sequencing errors in the L1 ORF (10). The nucleotide sequences of the L1 genes contained in clones 114/B and 114/K is shown in SEQ ID Nos. 1 and 2. A nucleotide sequence comparison of said both clones with the prototype HPV16 L1 coding region is given in Table 1, below. Recombinant baculovirus stocks were generated by cotransfection with baculovirus DNA (Baculo-Gold, PharMingen, San Diego, Calif.), using lipofection (Gibco/BRL, Gaithersburg, Md.), and plaque purification was carried out by standard techniques (6).

TABLE 1
Sequence comparison of the HPV16 L1 coding region
(nucleotide 5637-7154). The sequence of the prototype HPV16
L1 coding region can be found in (11).

| nt no. | 6216 | 6240 | 6432 |
|---|---|---|---|
| aa no. | 194 | 202 | 266 |
| prototype | gtt | cat | act |
| | Val | His | Thr |
| 114/K | gtt | gat | act |
| | Val | Asp | Thr |
| 114/B | att | gat | gct |
| | Ile | Asp | Ala |

nt: nucleotide; aa: amino acid
Underlining indicates differences from the prototype.

### EXAMPLE 2

### Generation of the HPV16 L2 Rabbit Antiserum

Sequences encoding a Glutathione-S-Transferase (GST)-HPV16 L2 fusion protein were constructed by PCR amplification of the entire L2 ORF of the prototype, using the same primers described above, and insertion of the resulting BglII fragment into the BamHI cloning site of the vector pGEX-2T (Pharmacia, Milwaukee, Wis.). The fusion protein was expressed in bacteria according to the specifications of the manufacturer, purified by preparative SDS-PAGE, and used to immunize a rabbit as described in (6).

### EXAMPLE 3

### Characterization of Proteins Expressed in Insect Cells

Sf-9 cells were mock infected or infected at a multiplicity of infection (MOI) of −10 with either wild type or recombinant baculovirus. After 72 hours, cells were lysed by boiling in SDS sample buffer and analyzed by SDS-PAGE in 10% gels. Proteins were stained with 0.25% Coomassie blue or analyzed by Western blotting, using the monoclonal antibody CAMVIR-1 (anti-HPV16 L1, PharMingen) or the GST-16L2 antiserum, followed by ¹²⁵I-labeled anti-mouse IgG or anti-rabbit IgG, respectively. The respective 58 kD L1 protein encoded by the three L1 genes (114/K, 114/B, prototype) were expressed at similar levels by the single and double expression viruses, as demonstrated by Coomassie staining of whole cell lysates and their reactivity to an anti-HPV16 L1 monoclonal antibody. Differences in the patterns of the more rapidly migrating immunoreactive peptides suggested that the prototype L1 protein may differ somewhat from the L1 proteins of the condylomata derived strains in its susceptibility to proteolytic cleavage.

L2 protein was not detected by Coomassie staining, whether expressed from the synthetic promoter or the polyhedrin promoter. However, Western blotting with a rabbit antiserum raised against a bacterially derived HPV16 L2 fusion protein detected a unique −90 kD protein in lysates from cells infected with the L2 single expressor or with the L1/L2 double expressing viruses, but not in lysates from control cells or from cells expressing L1 alone. Faster migrating immunoreactive bands probably represent proteolytic degradation products of the L2 protein. As expected, more L2 protein was detected in cells infected with the single expression vector, since the L2 gene in this vector is under control of the polyhedrin promoter. The lack of detectable Coomassie stained L2 protein suggests that the steady state level of L2 protein, expressed from the same promoter, is lower than that of L1 protein. The relative levels of L1 and L2 protein expressed from the L1/L2 double expressing viruses in the insect cells appears to be similar to the ratio of these two proteins in native PV virions (unpublished results).

### EXAMPLE 4

### Preparative Purification of Particles

For production of VLP, Sf-9 cells were grown at 27° C. as 500 ml suspension cultures in 1 liter spinner flasks (Bellco, Vineland, N.J.), using Grace's supplemented insect medium with 10% fetal calf serum (FCS), penicillin/streptomycin and 0.1% pluronic F-68 (all from Gibco/BRL, Gaithersburg, Md.). One liter of cells at a density of 3-3.5×10⁶/ml were harvested by low speed centrifugation and infected at a high MOI in 100 ml Grace's medium without FCS for 1 hour with periodical inversion. The cells were then grown as adherent cultures by plating them into twenty 245×245 mm tissue culture plates (Nunc, Naperville, Ill.) in a volume of 90 ml/plate Grace's medium with 10% FCS. After 72 hours, the cells were harvested by scraping and centrifuged at 2000 rpm (Sorvall RC-3B) for 5 min., washed once with ice cold PBS, and the final pellet was either snap frozen in liquid nitrogen for storage at −70° C. processed immediately. All subsequent procedures were carried out at 4° C. The cell pellet was resuspended in one volume PBS to a total of 24 ml, sonicated on ice twice for 45 seconds at setting 60% (Fisher Sonic Dismembrator), and the total cell lysate loaded on top of six 40% sucrose/PBS cushions and centrifuged in a SW-28 rotor at 25,000 rpm (=110,000 g) for 2.5 hours. Each of the resulting pellets was resuspended in 2 ml 27% CsCl in PBS by short pulse sonication, pooled into two quick seal tubes and centrifuged to equilibrium in 27% CsCl/PBS for 20 hours at 28,000 rpm (=141,000 g) in a SW-28 rotor. The visible band (at a density of −1.29 g/ml) was harvested by puncturing the tubes with an 18 gauge needle, centrifuged again using the identical conditions, dialyzed extensively against PBS, and stored at 4° C.

### EXAMPLE 5

### Detection and Quantification of VLPs by Transmission Electron Microscopy (TEM)

For TEM, particles were absorbed to carbon coated grids, stained with 1% uranyl acetate, and examined with a Philips electron microscope (model EM 400T) at 36,000× magnification as previously described (6).

To determine if the HPV16 L1 genes derived from productive lesions encode L1 proteins with the capacity to efficiently self-assemble, lysates from insect cells infected with each of the three L1 single expression baculoviruses (prototype, 114/K, 114/B) were subjected to cesium chloride density gradient centrifugation, and the visible band (at a density of −1.29 g/ml) was analyzed by TEM (see Example 4). As previously reported (6), only rare particles were seen with preparations of the prototype L1. By contrast, the L1 protein from clones 114/K and 114/B self-assembled into VLP with high efficiency, yielding several orders of magnitude more VLP than preparations of the prototype L1 protein, as estimated by the number of particles seen by TEM. The predominant structure consisted of spherical particles −50 nm in diameter with a regular array of capsomeres, but smaller, larger and irregular spheres were also seen as well as tubular structures. Within the resolving limits of the TEM, all the particles appeared to have the same subunit structure. Furthermore, there was a quantitative difference in the preparations from cells infected with the L1/L2 expressing virus compared to a virus expressing the L1 protein alone. Coexpression of the prototype L2 gene with the condylomata derived L1 genes (114/K, 114/B) resulted in a two-fold increase in particle yield and did not noticeably change the size or shape distribution of the particles. However, coexpression of the L2 gene did not increase the efficiency of prototype L1 assembly.

### EXAMPLE 6

### Analytical Gradient Centrifugation of Particles: Incorporation of L2 Protein Into VLPs When Coexpressed With L1 Protein

To determine if L2 protein was incorporated into VLP, 114K-L1 and the prototype L2 gene was coexpressed in insect cells from a double expression recombinant virus. VLP was purified as described in Example 4. As controls, L1 and L2 genes were expressed alone. Following purification in two sequential CsCl gradients, the L1, L2, and L1/L2 protein preparations were separated on concurrently run analytical sucrose gradients, and the resulting gradient fractions were analyzed by Western blotting, using antibodies specific for L1 or L2 protein, as well as by TEM.

The density profile of the three gradients was very similar, as determined by the refractive index. A protein with the expected −90 kD size was specifically recognized by the L2 antiserum in a dense gradient fraction (#2) of the L1/L2 double expressing preparation but not in the preparation that expressed only the L1 protein. The fraction with the L2 protein also contained a large proportion of the immunoreactive L1 protein, as well as many VLP by TEM. These results demonstrate that the prototype L2 gene is incorporated into VLP when coexpressed with 114/K-L1 in insect cells and that encapsidation by L1 protein may protect it from proteolytic degradation.

The analytical gradient centrifugation and the detection of L2 protein in VLPs was carried out as follows: a 12-45% sucrose step gradient was allowed to linearize overnight at 4° C., dialyzed samples were layered on top, and the gradient centrifuged at 41,000 rpm for 3 hours in a SW-41 rotor (=288,000 g). Factions were harvested from the bottom and analyzed by TEM and Western blotting using monoclonal antibody CAMVIR-1 or the GST-16 L2 rabbit antiserum. The densities of the fractions were calculated from the refractive index (20° C.), as determined by an Abba-3L refractometer (Milton Roy, Rochester, N.Y.).

### LITERATURE

(1) Kreider; in: Viruses in naturally occurring cancers, M Essex, G Todaro and H zur Hausen (eds), Cold Spring Harbor Laboratory, Cold Spring Harbor, N.Y. (1980), 283-299

(2) Camp et al.; Cancer Research 52 (1992), 1-7

(3) Campo and Jarret; Ciba Foundation Symp. 120, (1986), 117-135

(4) Gissmann; Seminars in Cancer Biology 3 (1992), 253-261

(5) Christensen et al.; J. Virol. 64 (1990), 5678-5681

(6) Kirnbauer et al.; Proc. Natl. Acad. Sci. USA 89 (1992), 12180-12184

(7) Zhou et al.; J. Virol. 185 (1991), 251-257

(8) Wang et al.; Gene 100 (1991), 131-137

(9) Halbert et al.; J. Virol. 62 (1988), 1071-1075

(10) Parton et al.; Nucl. Acids Res. 18 (1990), 3631

(11) Seedorf et al.; Virology 145 (1985), 181-185

1. A DNA sequence encoding an L1 protein of a papillomavirus capable of forming VLPs which is
(a) the DNA sequence of DNA ID No 1 or 2 or a part thereof containing triplett 202;
(b) a DNA sequence hybridizing to the sequence of (a) comprising at triplett 202 a DNA sequence encoding Asp or Glu; or
(c) a DNA sequence which is related to the DNA sequence of (a) or (b) by degeneration of the genetic code.

2. The DNA sequence of claim 1 which is derived from HPV16 (DSM 8419) or HPV16 (DSM 8418) and encodes an L1 protein.

3. A recombinant vector comprising the DNA sequence of claim 1 or 2.

4. The recombinant vector of claim 3 wherein said DNA sequence is under the control of regulatory elements allowing its expression in a desired host cell.

5. A host cell transformed with the recombinant vector of claims 3 or 4.

6. The host cell of claim 5 which is an insect cell, a bacterial cell, a yeast cell, a mammalian cell or a plant cell.

7. A method of producing an L1 protein capable of forming VLPs comprising the cultivation of a host cell according to claim 5 or 6 under conditions appropriate for the expression of said DNA sequences and recovering said protein from the culture.

8. An L1 protein encoded by the DNA sequence of claim 1 or 2.

9. An L1 protein produced by the method of claim 7.

10. A VLP comprising the L1 protein of claim 8 or 9.

11. The VLP of claim 10 which additionally comprises an L2 protein.

12. An antibody which is specifically directed against the VLP of claim 10 or 11 or the L1 protein of claim 8 or 9.

13. The antibody of claim 12 which is a monoclonal antibody.

14. A pharmaceutical composition comprising the VLP of claim 10 or 11, optionally in combination with a pharmaceutically acceptable carrier and/or diluent.

15. The pharmaceutical composition of claim 14 which is a vaccine.

16. The pharmaceutical composition of claim 15 which is a vaccine against papillomavirus infections.

17. The pharmaceutical composition of claim 16, wherein said papillomavirus infections is an HPV16 infection.

18. A diagnostic kit for the measurement of anti-HPV16 virion antibodies comprising the VLP of claim 10 or 11.

19. A method for determining anti-HPV16 virion antibodies in a sample comprising the use of the VLP of claim 10 or 11 as antigen.

20. A method for the prophylaxis of papillomavirus infections comprising administering an effective dosage of the VLP of claim 10 or 11 to a patient in need thereof.


SEQUENCE LISTING (6 sequences)

SEQ ID NO: 1 - DNA, 1518 bp, Artificial Sequence
  Description of Artificial Sequence Nucleotide sequence of L1 gene of HPV16 DNA clone P114/16/2
atgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacaattattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaa

SEQ ID NO: 2 - DNA, 1518 bp, Artificial Sequence
  Description of Artificial Sequence Nucleotide sequence of L1 gene of HPV16 DNA clone P114/16/11
atgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaa

SEQ ID NO: 3 - protein, 505 aa, Artificial Sequence
  Description of Artificial Sequence Amino acid sequence of L1 gene of HPV16 clone
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTIIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 4 - protein, 505 aa, Artificial Sequence
  Description of Artificial Sequence Amino acid sequence of L1 gene of HPV16 clone
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGTVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 5 - DNA, 50 bp, Artificial Sequence
  Description of Artificial Sequence Primer
gcggtgatatcaatatgcgacacaaacgttctgcaaaacgcacaaaacgt

SEQ ID NO: 6 - DNA, 44 bp, Artificial Sequence
  Description of Artificial Sequence Primer
ccgctccgcggactgggacaggaggcaagtagacagtggcctca